SEQUENCE LISTING (18 sequences)

SEQ ID NO: 1 - protein, 523 aa, Canavalia ensiformis
ATLKSIIEPTESLTYLWPLPADFTSGDETLSVDPALTLSVAGNGGGSSILRDAFDRYRGIIFKHSSVGFSLIRKLRERLVSVSAYDIATLKITVHSDNEELQLGVDETYTLLVPKAKDSYVAGEVTIEANTVYGALRGLETFSQLCSFDYSDKTIKIYKAPWSIQDKPRFSYRGLLLDTSRHYLPINVIKQIIESMSYAKLNVLHWHIIDEESFPLEVPTYPNLWKGSYTKWERYTVEDAYEIVNFAKMRGINVMAEVDVPGHAESWGAGYPNLWPSPSCREPLDVSKNFTFDVISGILTDIRKIFPFELFHLGGDEVNTDCWTSTSHVKEWLSTQNMTAKDAYEYFVLKAQEIAVSKNWSPVNWEETFNTFPAKLHKKTVVHNWLGPGVCPKVVAKGFRCIFSNQGVWYLDHLDVPWDEVYTAEPLEGIEKSSEQELVIGGEVCMWGETADTSNVQQTIWPRAAAAAERLWSQRDSTNITVTALPRLQNFRCLLNKRGVAAAPVKNYYARRAPSGPGSCYEQ

SEQ ID NO: 2 - DNA, 1569 bp, Canavalia ensiformis
gctactttgaagtccatcatcgagccaactgagtccttgacttacttgtggccattgccagctgacttcacttctggtgacgaaactttgtctgttgacccagctttgactttgtccgttgctggtaatggtggtggttcctccattttgagagatgctttcgacagatacagaggtattatcttcaagcactcctccgttggattctctttgatcagaaagttgagagagagattggtttccgtttccgcttacgacattgctactttgaagatcactgttcactccgacaacgaagagttgcagttgggtgttgacgagacttacactttgttggttccaaaggctaaggactcctacgttgctggtgaggttactatcgaggctaacactgtttacggtgctttgagaggtttggagactttctcccagttgtgttccttcgactactctgacaagactatcaagatttacaaggctccttggtccatccaggacaagccaagattttcctacagaggtttgttgttggacacttccagacactacttgccaatcaacgttatcaagcagatcatcgagtccatgtcctacgctaagttgaacgttttgcactggcacatcatcgacgaagagtctttcccattggaggttccaacttacccaaacttgtggaagggttcctacactaagtgggagagatacactgttgaggacgcttacgagatcgttaacttcgctaagatgagaggtattaacgttatggctgaggttgacgttccaggtcatgctgaatcttggggtgctggttatccaaatttgtggccatctccatcctgtagagagccattggacgtttccaagaacttcactttcgacgttatctccggaatcttgactgacatcagaaagatattcccattcgagttgttccacttgggaggtgacgaggttaatactgactgttggacttccacttcccacgttaaggaatggttgtccactcagaacatgactgctaaggatgcttacgaatacttcgttttgaaggctcaagagatcgctgtttctaagaactggtcccctgttaactgggaagagactttcaacactttcccagctaagttgcacaagaaaactgttgttcacaactggttgggtccaggtgtttgtccaaaggttgttgctaagggtttcagatgtatcttctccaaccagggtgtttggtacttggaccacttggatgttccttgggacgaggtttacactgctgaaccattggaaggtatcgagaagtcctctgagcaagagttggttatcggtggtgaagtttgtatgtggggtgagactgctgacacttctaacgttcagcagactatctggccaagagccgcagctgctgctgaaagattgtggtcccaaagagactccactaacatcactgttactgctttgccaagattgcagaacttcagatgtttgttgaacaagagaggtgttgctgctgctccagttaagaactactacgctagaagagccccatccggtccaggttcttgttacgaacaa

SEQ ID NO: 3 - DNA, 26 bp, Artificial Sequence
  Primer JB-01
ctcacctacctctggccccttcccgc

SEQ ID NO: 4 - DNA, 33 bp, Artificial Sequence
  Primer JB-07
ttattggtcataacatgaccctggaccaacagg

SEQ ID NO: 5 - DNA, 25 bp, Artificial Sequence
  Primer JB-02
gaggagcttcaatttggagtggatg

SEQ ID NO: 6 - DNA, 32 bp, Artificial Sequence
  Primer JB-06
atcagctgtctcaccccacatgcaaacttctc

SEQ ID NO: 7 - DNA, 22 bp, Artificial Sequence
  Primber JB-08
aagtttgcatgtggggtgagac

SEQ ID NO: 8 - DNA, 23 bp, Artificial Sequence
  Primer JB-09
gcaaacaatatggcctagagctg

SEQ ID NO: 9 - DNA, 28 bp, Artificial Sequence
  CDSIII-short
attctagaggccgaggcggccgacatgt

SEQ ID NO: 10 - DNA, 22 bp, Artificial Sequence
  JB-10
aagagtccttggctttgggaac

SEQ ID NO: 11 - DNA, 35 bp, Artificial Sequence
  Okib57-Adapter
gtaggaattcgggttgtagggaggtcgacattgcc

SEQ ID NO: 12 - DNA, 22 bp, Artificial Sequence
  JB-11
tcaatgtcgcaatgtcataggc

SEQ ID NO: 13 - DNA, 22 bp, Artificial Sequence
  JB-12
atgagactgaacccaacactgc

SEQ ID NO: 14 - DNA, 23 bp, Artificial Sequence
  Okib58
ggcaatgtcgacctccctacaac

SEQ ID NO: 15 - DNA, 23 bp, Artificial Sequence
  Okib59
ctccctacaacccgaattcctac

SEQ ID NO: 16 - protein, 553 aa, Canavalia ensiformis
MFLCIPRWFSSPLLILFVIYCALFAPQAASATLKSIIEPTESLTYLWPLPADFTSGDETLSVDPALTLSVAGNGGGSSILRDAFDRYRGIIFKHSSVGFSLIRKLRERLVSVSAYDIATLKITVHSDNEELQLGVDETYTLLVPKAKDSYVAGEVTIEANTVYGALRGLETFSQLCSFDYSDKTIKIYKAPWSIQDKPRFSYRGLLLDTSRHYLPINVIKQIIESMSYAKLNVLHWHIIDEESFPLEVPTYPNLWKGSYTKWERYTVEDAYEIVNFAKMRGINVMAEVDVPGHAESWGAGYPNLWPSPSCREPLDVSKNFTFDVISGILTDIRKIFPFELFHLGGDEVNTDCWTSTSHVKEWLSTQNMTAKDAYEYFVLKAQEIAVSKNWSPVNWEETFNTFPAKLHKKTVVHNWLGPGVCPKVVAKGFRCIFSNQGVWYLDHLDVPWDEVYTAEPLEGIEKSSEQELVIGGEVCMWGETADTSNVQQTIWPRAAAAAERLWSQRDSTNITVTALPRLQNFRCLLNKRGVAAAPVKNYYARRAPSGPGSCYEQ

SEQ ID NO: 17 - DNA, 1662 bp, Canavalia ensiformis
atgtttctgtgcatacccagatggttctcttcacctcttctcattctctttgtcatttactgtgccctctttgctcctcaagctgcttctgccacactcaaatctatcattgaacccactgagtccctcacatacctttggcccctccccgcagacttcacttcaggcgatgaaactctttccgttgaccctgcacttaccctctctgtcgccggcaacggtggtggctcttccattctcagagatgcatttgaccgatacagaggaatcatattcaagcacagcagtgttgggttcagtctcataagaaagttaagggaaagattggtgtctgtttctgcctatgacattgcgacattgaagatcactgtccattcagataacgaggagcttcaacttggagtggatgaaacctataccttgctggttcccaaagccaaggactcttatgttgctggggaagtcacaattgaggcaaacactgtttatggtgcattgcgcggattagagacattcagccagttgtgttctttcgattattcggataaaacaataaaaatatacaaggcaccttggtccatccaagataaacctagattttcctatcgtgggcttttgttggacacatcgaggcactatttaccaattaacgtaattaagcagattattgaatctatgtcctatgctaaacttaatgttctacattggcacatcatagacgaggagtcatttcctcttgaggtacctacatatccaaacttgtggaaaggttcatatacaaagtgggaacgttacacggtagaagacgcatatgaaattgtcaacttcgccaaaatgagaggcataaatgtgatggcagaagtggatgttcctggtcatgcagaatcatggggtgctggatatcccaatctttggccgtcaccttcctgtagggagccactggatgtttcaaagaattttacttttgatgtcatttctggtatcctgacagatataagaaagattttcccgtttgagctatttcacttgggtggtgatgaagttaatacagattgctggaccagtacttctcatgtgaaggaatggctttcgactcaaaacatgactgctaaagatgcctatgaatattttgtactgaaggcccaagagatagctgtttcaaaaaattggagtccggtgaactgggaagaaaccttcaatacatttccagcaaagctccataagaaaactgtggtgcataactggttgggccctggggtttgtccaaaggttgttgcaaaaggtttcaggtgcattttcagtaatcagggtgtctggtatcttgaccatctggatgtaccttgggatgaggtctatactgctgagccactagaaggaatagaaaaatcttctgaacaagagcttgtaattggaggagaagtttgcatgtggggtgagacagctgatacatccaatgttcagcaaacaatatggcctagagctgctgcagctgcagaacgcttatggagtcagagagattctacaaatattactgtaactgcgttgccccggttacaaaacttcagatgtctattgaataaacgtggagttgcagctgctcctgtgaaaaattattatgctagaagggctcctagtggtccaggctcatgttatgagcaataa

SEQ ID NO: 18 - DNA, 1572 bp, Artificial Sequence
  codon optimized sequence (for human cells)
gccacactgaagtccatcatcgagcccaccgagagcctgacctacctgtggcctctgcccgccgatttcaccagcggcgacgagacactgtccgtggatcctgccctgacactgagcgtggccggaaatggcggcggaagcagcatcctgagagatgccttcgaccggtacagaggcatcatcttcaagcacagcagcgtgggcttcagcctgatccggaagctgcgcgagagactggtgtccgtgtccgcctacgatatcgccaccctgaagatcaccgtgcactccgacaacgaggaactgcagctgggcgtggacgagacatacaccctgctggtgcccaaggccaaggacagctatgtggccggcgaagtgaccatcgaggccaacacagtgtacggcgccctgagaggcctggaaaccttcagccagctgtgcagcttcgactacagcgacaagaccatcaagatctacaaggccccttggagcatccaggacaagccccggttcagctacagaggcctgctgctggacaccagcagacactacctgcccatcaacgtgatcaagcagatcatcgagagcatgagctacgccaagctgaacgtgctgcactggcacatcatcgacgaggaatccttcccactggaagtgcccacctaccccaacctgtggaagggcagctacaccaagtgggagcggtacaccgtggaagatgcctacgagatcgtgaacttcgccaagatgcggggcatcaatgtgatggccgaggtggacgtgccaggccacgctgaatcttggggagccggctaccctaatctgtggcccagccccagctgtcgcgaacccctggacgtgtccaagaacttcaccttcgacgtgatcagcggcatcctgaccgatatcagaaagatcttcccattcgagctgttccacctgggaggcgacgaagtgaacaccgactgctggaccagcaccagccacgtgaaagagtggctgagcacccagaacatgaccgccaaggacgcctacgagtacttcgtgctgaaggcccaggaaatcgccgtgtctaagaattggagccccgtgaactgggaggaaacctttaacaccttccctgccaaactgcacaagaaaaccgtggtgcacaattggctgggccctggcgtgtgccctaaggtggtggccaagggcttccgctgcatattcagcaaccagggcgtgtggtatctggaccacctggatgtgccctgggacgaggtgtacacagccgagcctctggaaggcatcgagaagtcctccgagcaggaactcgtgatcggcggagaagtgtgcatgtggggcgagacagccgacacctccaacgtgcagcagaccatctggcctagagccgccgctgccgctgaaagactgtggtcccagagagacagcaccaacatcaccgtgaccgccctgccccggctgcagaactttagatgcctgctgaacaagcggggcgtggccgctgcccccgtgaagaattactatgccagaagggcccccagcggccctggcagctgttatgaacagtga

(19) United States
(12) Patent Application Publication
(10) Pub. No.: US 2024/0110168 A1
(43) Pub. Date: Apr. 4, 2024

(54) Polypeptide Having Beta-Hexosaminidase Activity, and Polynucleotides Coding for the Same

(71) Applicant: Genzyme Corporation, Cambridge, MA (US)

(72) Inventors: Sebastian RISSOM, Frankfurt am Main (DE); Ava CHATTOPADHYAY, Frankfurt am Main (DE); Werner DITTRICH, Frankfurt am Main (DE); Thomas Martin WENDRICH, Frankfurt am Main (DE)

(73) Assignee: Genzyme Corporation, Cambridge, MA (US)

(21) Appl. No.: 18/255,832

(22) PCT Filed: Dec. 2, 2021
(86) PCT No.: PCT/EP2021/084004

§ 371 Date: Jun. 2, 2023

Related U.S. Application Data
(60) Provisional application No. 63/120,608, filed on Dec. 2, 2020.

(30) Foreign Application Priority Data
Dec. 11, 2020 (EP) 20213521.6

(51) Int. Cl. C12N 9/24

(52) U.S. Cl. CPC C12N 9/2402; C12Y 302/01052

(57) ABSTRACT

The present invention relates to a method of producing a polypeptide having β-hexosaminidase activity, comprising the steps of a) providing a yeast cell comprising a polynucleotide encoding a polypeptide having β-hexosaminidase activity and having an amino acid sequence being at least 95% identical to the amino acid sequence shown in SEQ ID NO: 1, b) cultivating said yeast cell under conditions which allow for the production of the polypeptide, and c) obtaining the polypeptide produced in step b). The present invention further concerns a polynucleotide encoding a polypeptide having β-hexosaminidase activity and having an amino acid sequence being at least 95% identical to the amino acid sequence shown in SEQ ID NO: 1, as well as polypeptide encoded by said polynucleotide. Moreover, the present invention concerns a yeast cell comprising the polynucleotide of the present invention.

### FIELD OF THE INVENTION

The present invention relates to a method of producing a polypeptide having β-hexosaminidase activity, comprising the steps of a) providing a yeast cell comprising a polynucleotide encoding a polypeptide having β-hexosaminidase activity and having an amino acid sequence being at least 95% identical to the amino acid sequence shown in SEQ ID NO: 1 or 16, b) cultivating said yeast cell under conditions which allow for the production of the polypeptide, and c) obtaining the polypeptide produced in step b). The present invention further concerns a polynucleotide encoding a polypeptide having β-hexosaminidase activity and having an amino acid sequence being at least 95% identical to the amino acid sequence shown in SEQ ID NO: 1, as well as polypeptide encoded by said polynucleotide. Moreover, the present invention concerns a yeast cell and a vector comprising the polynucleotide of the present invention.

### BACKGROUND

β-hexosaminidase (EC 3.2.1.52, abbreviated “b-Hex” herein) is an enzyme that catalyzes the hydrolysis of terminal nonreducing N-acetylhexosamine residues in N-acetyl-beta-hexosaminides. The enzyme is frequently also referred to as N-acetyl-beta-glucosaminidase. N-Acetylglucosides and N-acetylgalactosides are substrates.

Three major forms of the β-hexosaminidase are found in mammals: a trimer consisting of one alpha chain, one beta-A chain, and one beta-B chain (Form A), a tetramer consisting of two beta-A chains and two beta-B chains (Form B), and a homodimer of two alpha chains (Form S). It is known that some genetic disorders such as Tay-Sachs disease and Sandhoff's disease result from mutations in the human b-Hex gene.

Glycosidases have been used as tools in glycobiology research for decades, and their role in glycoprotein maturation has been studied (as reviewed by Léonard R, Strasser R, Altmann F. Plant glycosidases acting on protein-linked oligosaccharides. Phytochemistry. 2009 February; 70(3):318-24. doi: 10.1016/j.phytochem. 2009.01.006. Epub 2009 Feb. 4. PMID: 19200565).

A β-hexosaminidase enzyme preparation currently used for glycan modification is extracted from its natural source Jack Beans (Canavalia ensiformis). The basic description of the enzyme and of the base of the current extraction process can be found in Li et al. (J. Biol. Chem. 1970 245: 5153-5160). The enzyme has been used, for example, to study enzymatic detachment of biofilms (J Med Microbiol. 2006 August; 55(Pt 8):999-1008).

This current extraction process however has several disadvantages:

Jack Bean being a plant grown on fields has the disadvantages of such natural systems: High level of irreproducibility due to weather, soil, etc. conditions (see Li (1970)). As consequence new secondary metabolites could arise, which then introduced into the production process might finally contaminate the drug substance with incalculable effects on the patient health. Use of agrochemicals to maintain fertility and to avoid damage to plants can lead to residues within the product. Presence of fungi or other microbial contaminants in the soil, on the plants or on the beans during culture or storage can lead to the contamination of the product by toxins such as aflatoxins that can have a very toxic effect even in small amounts.

B-Hex is not a very abundant protein in Jack Beans as the plant does not need high quantities of this enzyme under natural conditions. It is present in only very small activities of about 1 U/g of bean material and thus needs to be extracted and subsequently separated and purified from a large number and quantity of contaminating proteins. This difficult procedure results in a high need of plant material to be used as substrate and makes this process very costly.

Except for the few data described for b-Hex described in early 1970s (see Li et al., cited above), there is not a lot known about this enzyme. Notably, there is no protein or DNA sequence publicly available. No detailed structural characterization of the enzyme has been made.

Gers-Barlag et al. describe the isolation of the β-hexosaminidase from soybean (Phytochemistry, Volume 27, Issue 12, 1988, Pages 3739-3741). US 2004/0031072 discloses the sequence of the β-hexosaminidase from soybean (as SEQ ID NO: 162900). Soybean β-hexosaminidase sequences can be also assessed via UniProt (see accession number I1KTU6 or I1JDS6 which corresponds to NCBI Reference Sequence: XP 003518662.1).

CN 109 971 736 describes the identification of an hexosaminidase enzyme from strawberry.

Slámová et al. describe the cloning and high-yield expression of a fungal b-N-acetylhexosaminidase in Pichia pastoris (Protein Expr Purif. 2012 March; 82(1):212-7. doi: 10.1016/j.pep.2012.01.004. Epub 2012 Jan. 11).

Strasser describes the heterologous expression of three putative b-Hex sequences present in the Arabidopsis (Arabidopsis thaliana) genome (Strasser et al., Plant Physiol. 2008 June; 147(2):931). The authors used a Spodoptera frugiperda Sf21 insect cell system for expression. The authors also showed that these plant enzymes have only very limited homology of around 30% with the well-studied human b-Hex enzymes HexA and HexB. It is thus not astonishing that the microbial expression in the methylotrophic yeast Ogataea minuta described in Akeboshi et al. for the human HexA would not be transposable to plant enzymes (Akeboshi et al., Appl Environ Microbiol. 2007 August; 73(15):4805-12). This is especially true as the authors describe two major differences between the recombinant HexA from O. minuta and native HexA from human lysosomes corresponding to different post-translational processing between both organisms.

### SUMMARY OF THE PRESENT INVENTION

In the context of the studies underlying the present application, a detailed analysis of the b-Hex enzyme isolated from Jack Bean (Canavalia ensiformis) was carried out in order to determine as much of the protein sequence as possible. This was done by applying a combination of protease digestion, Edman sequencing and LC-MS/MS analyses, and led to about 40% sequence coverage of the protein (Example 2). With this result, it was possible to confirm by data base search that effectively no match to the found sequence was available. The closest sequences found belong to b-Hex proteins from Soy Bean (Glycine max). Further, the full length cDNA sequence coding for the Jack Bean b-Hex enzyme was determined (Example 3). The alignment with sequences available in databases revealed no known sequence to match the one determined, thus the detected β-hexosaminidase polypeptide seems to be not known to the public domain.

Advantageously, it was possible to express the β-hexosaminidase polypeptide in a microbial system, namely in Komagataella phaffii (sometimes also referred to as Pichia pastoris). Supernatants of cultures containing the generated Komagataella phaffii strains showed a significant amount of b-Hex activity (Example 4). It was shown that more than 100 U/mL of culture can be achieved. Additionally, the culture supernatant did not contain major amounts of contaminating proteins. This allows a straight-forward reproducible protein purification process.

The Klyveromyces lactis expression system described by Swennen (2002) was chosen as a second example. The recombinant expression of b-Hex was also successful for this yeast system as biologically active b-Hex was found in the respective yeast cultures.

It was shown that the b-Hex enzyme from Jack Bean is not present in a single polypeptide chain as it would be expected by translating the found DNA sequence into a protein sequence. It was rather found that there are two polypeptide chains associated with each other without being covalently linked. Dissociation of the two chains led to a complete loss of activity. Therefore, it was surprising that the recombinant microbial expression yielded active enzyme, since it was not expected that the identified b-Hex enzyme would reproduce such split into two chains and in the same time ensure that these two chains are associated in the correct structure.

Accordingly, the present invention relates to a method of producing a polypeptide having β-hexosaminidase activity, comprising the steps of
a) providing a host cell comprising a polynucleotide encoding a polypeptide having β-hexosaminidase activity and having an amino acid sequence being at least 85% identical to the amino acid sequence shown in SEQ ID NO: 1 or 16,
b) cultivating said host cell under conditions which allow for the production of the polypeptide, and
c) obtaining the polypeptide produced in step b).

The present invention further relates to a polynucleotide encoding a polypeptide having β-hexosaminidase activity and having an amino acid sequence being at least 85% identical to the amino acid sequence shown in SEQ ID NO: 1 or 16.

Further encompassed by the present invention is an isolated polypeptide encoded by the polynucleotide of the present invention.

Moreover, the present invention is directed to a vector comprising the polynucleotide of the present invention. In some embodiments, the vector is an expression vector.

The present invention further pertains to a host cell comprising the polynucleotide of the present invention, the polypeptide of the present invention and/or the vector of the present invention.

In some embodiments, the host cell of the present invention is a yeast cell or animal cell. For example, the host cell may be a yeast cell belonging to the family of Saccharomycetaceae, such as a Komagataella phaffii cell.

In some embodiments, the polypeptide of the present invention has an amino acid sequence which is at least 90% identical, such as 95% or 98% identical, to the amino acid sequence shown in SEQ ID NO: 1 or 16. In some embodiments, the polypeptide comprises an amino acid sequence as shown in SEQ ID NO: 1 or 16.

In some embodiments, the polynucleotide of the present invention comprises a nucleic acid sequence as shown in SEQ ID NO: 2. In some embodiments, the polynucleotide of the present invention comprises a nucleic acid sequence as shown in SEQ ID NO: 17.

In some embodiments, the polynucleotide of the present invention is operably linked to a heterologous promoter.

In some embodiments, the polynucleotide of the present invention is codon optimized for the host cell, such as a yeast cell.

### DETAILED DESCRIPTION OF THE PRESENT INVENTION

### Definitions

As set forth above, the present invention relates to a method of producing a polypeptide having β-hexosaminidase activity, comprising the steps of
a) providing a host cell comprising a polynucleotide encoding a polypeptide having β-hexosaminidase activity and having an amino acid sequence being at least 85% identical to the amino acid sequence shown in SEQ ID NO: 1 or 16,
b) cultivating said host cell under conditions which allow for the production of the polypeptide, and
c) obtaining the polypeptide produced in step b).

In step a) of the method of the present invention, a host cell shall be provided comprising a polynucleotide encoding a polypeptide having β-hexosaminidase activity.

The term “polynucleotide” as used herein refers to a linear or circular nucleic acid molecule. It encompasses DNA as well as RNA molecules. The polynucleotide shall be provided either as an isolated polynucleotide (i.e. isolated from its natural context) or in genetically modified form. The polynucleotide as set forth herein is characterized in that it shall encode a polypeptide as referred to above, i.e. a polypeptide having β-hexosaminidase activity.

The terms “polypeptide” and “protein” are used interchangeably herein and refer to amino acids in a polymeric form linked together by peptide bonds.

The polypeptide produced by the method of the present invention shall have β-hexosaminidase activity.

As used herein, β-hexosaminidase (EC 3.2.1.52) typically refers to an enzyme being capable of catalysing the hydrolysis of terminal nonreducing N-acetylhexosamine residues in N-acetyl-beta-hexosaminides. For example, N-acetylglucosides and N-acetylgalactosides are substrates. Assays for assessing whether a polypeptide has β-hexosaminidase activity are known in the art and described, for example, in Li & Li (1970) J Biol Chem 245 5153: They show b-hexosaminidase activity for following substrates: p-nitrophenyl β-2-acetamido-2-deoxy-p-glucopyranoside and p-nitrophenyl β-2-acetamido-2-deoxy-p-galactopyranoside. Synonyms are β-hexosaminidase, β-(1-2,3,4,6) Hexosaminidase; β-acetylamino-deoxyhexosidase, N-acetyl-β-D-hexosaminidase; N-acetyl-β-hexosaminidase; β-acetylhexosaminidinase, β-D-N-acetylhexosaminidase; β-N-acetyl-D-hexosaminidase, β-N-acetylglucosaminidase, N-acetylhexosaminidase and β-D-hexosaminidase.

In some embodiments, the polypeptide having β-hexosaminidase activity forms a homodimer.

In some embodiments, the polypeptide having β-hexosaminidase activity is expressed from a heterologous polynucleotide, i.e. from a polynucleotide which has been either transiently, e.g., by using an expression vector, or stably introduced into the host cell. The term “heterologous” as used herein means that the polynucleotide does not occur naturally in the host cell. The term, thus, encompasses modified or unmodified polynucleotides which are derived from different organisms or modified polynucleotides derived from the host cell. It is to be understood that the heterologous polynucleotide may either comprise expression control sequences which allow for expression in the host cell or sequences which allow for integration of the heterologous polynucleotide at a locus in the genome of the host cell where the expression of the heterologous polynucleotide will be governed by endogenous expression control sequences of the host cell. By introducing the heterologous polynucleotide, a transgenic host cell is generated.

The introduction of the polypeptide having β-hexosaminidase activity may be achieved by introducing heterologous polynucleotides encoding the said polypeptide into the host cell. The term “introduction” or “transformation” as referred to herein encompasses the transfer of a polynucleotide as described herein into a host cell, irrespective of the method used for transfer. This includes transient introduction in expression vectors or stable integration into the genome of the host cell. In some embodiments, the polynucleotide is stably introduced into the genome of the host cell.

Thus, step a) of the method of the present invention may comprise the steps of:
a1) introducing the polynucleotide encoding the polypeptide having β-hexosaminidase activity into the host cell; and
a2) expressing said polypeptide from the said polynucleotide.

The term “expression” or “gene expression” means the transcription of a specific gene or specific genes or specific genetic construct. The term “expression” or “gene expression” in particular means the transcription of a gene or genes or genetic construct into structural mRNA with subsequent translation of the latter into a polypeptide as referred herein. The process includes transcription of DNA and processing of the resulting mRNA product.

As set forth above, the polypeptide encoded by the polynucleotide of the present invention shall have β-hexosaminidase activity. In addition, it shall have an amino acid sequence being at least 85%, 90%, 91%, 92%, 93%, 94%, 95%, 96%, 97%, 98%, 99% or 100% identical to the amino acid sequence shown in SEQ ID NO: 1 or 16.

In some embodiments, the polypeptide having β-hexosaminidase activity has an amino acid sequence which is at least 95% identical, such as at least 98% identical to the amino acid sequence shown in SEQ ID NO: 1 or 16.

In some embodiments, the polypeptide having β-hexosaminidase activity comprises an amino acid sequence as shown in SEQ ID NO: 16.

SEQ ID NO: 16 is the amino acid sequence of the Jack Bean (Canavalia ensiformis) β-hexosaminidase identified in the studies underlying the present invention. The sequence is as follows:

MFLCIPRWFS SPLLILFVIY CALFAPQAAS ATLKSIIEPT
ESLTYLWPLP ADFTSGDETL SVDPALTLSV AGNGGGSSIL
RDAFDRYRGI IFKHSSVGFS LIRKLRERLV SVSAYDIATL
KITVHSDNEE LQLGVDETYT LLVPKAKDSY VAGEVTIEAN
TVYGALRGLE TFSQLCSFDY SDKTIKIYKA PWSIQDKPRF
SYRGLLLDTS RHYLPINVIK QIIESMSYAK LNVLHWHIID
EESFPLEVPT YPNLWKGSYT KWERYTVEDA YEIVNFAKMR
GINVMAEVDV PGHAESWGAG YPNLWPSPSC REPLDVSKNF
TFDVISGILT DIRKIFPFEL FHLGGDEVNT DCWTSTSHVK
EWLSTQNMTA KDAYEYFVLK AQEIAVSKNW SPVNWEETFN
TFPAKLHKKT VVHNWLGPGV CPKVVAKGFR CIFSNQGVWY
LDHLDVPWDE VYTAEPLEGI EKSSEQELVI GGEVCMWGET
ADTSNVQQTI WPRAAAAAER LWSQRDSTNI TVTALPRLQN
FRCLLNKRGV AAAPVKNYYA RRAPSGPGSC YEQ

The sequence of a potential leaded sequence (aa 1 to 30) is underlined. In the studies underlying the present invention, the polypeptide was expressed without the leader sequence. SEQ ID NO: 1 is the amino acid sequence of the Jack Bean (Canavalia ensiformis) β-hexosaminidase without the leader sequence. Accordingly, SEQ ID NO: 1 comprises aa 31 to 553 of SEQ ID NO: 16. SEQ ID NO: 1 is as follows:

ATLKSIIEPT ESLTYLWPLP ADFTSGDETL SVDPALTLSV
AGNGGGSSIL RDAFDRYRGI IFKHSSVGFS LIRKLRERLV
SVSAYDIATL KITVHSDNEE LQLGVDETYT LLVPKAKDSY
VAGEVTIEAN TVYGALRGLE TFSQLCSFDY SDKTIKIYKA
PWSIQDKPRF SYRGLLLDTS RHYLPINVIK QIIESMSYAK
LNVLHWHIID EESFPLEVPT YPNLWKGSYT KWERYTVEDA
YEIVNFAKMR GINVMAEVDV PGHAESWGAG YPNLWPSPSC
REPLDVSKNF TFDVISGILT DIRKIFPFEL FHLGGDEVNT
DCWTSTSHVK EWLSTQNMTA KDAYEYFVLK AQEIAVSKNW
SPVNWEETFN TFPAKLHKKT VVHNWLGPGV CPKVVAKGFR
CIFSNQGVWY LDHLDVPWDE VYTAEPLEGI EKSSEQELVI
GGEVCMWGET ADTSNVQQTI WPRAAAAAER LWSQRDSTNI
TVTALPRLQN FRCLLNKRGV AAAPVKNYYA RRAPSGPGSC YEQ

In an embodiment, SEQ ID NO: 1 additionally comprises a methionine residue (M) at the N-terminal end.

In an embodiment, the above polypeptide is encoded by a polynucleotide comprising a nucleic acid sequence as shown in SEQ ID NO: 2:

gctactttgaagtccatcatcgagccaactgagtccttgacttacttgtg
gccattgccagctgacttcacttctggtgacgaaactttgtctgttgacc
cagctttgactttgtccgttgctggtaatggtggtggttcctccattttg
agagatgctttcgacagatacagaggtattatcttcaagcactcctccgt
tggattctctttgatcagaaagttgagagagagattggtttccgtttccg
cttacgacattgctactttgaagatcactgttcactccgacaacgaagag
ttgcagttgggtgttgacgagacttacactttgttggttccaaaggctaa
ggactcctacgttgctggtgaggttactatcgaggctaacactgtttacg
gtgctttgagaggtttggagactttctcccagttgtgttccttcgactac
tctgacaagactatcaagatttacaaggctccttggtccatccaggacaa
gccaagattttcctacagaggtttgttgttggacacttccagacactact
tgccaatcaacgttatcaagcagatcatcgagtccatgtcctacgctaag
ttgaacgttttgcactggcacatcatcgacgaagagtctttcccattgga
ggttccaacttacccaaacttgtggaagggttcctacactaagtgggaga
gatacactgttgaggacgcttacgagatcgttaacttcgctaagatgaga
ggtattaacgttatggctgaggttgacgttccaggtcatgctgaatcttg
gggtgctggttatccaaatttgtggccatctccatcctgtagagagccat
tggacgtttccaagaacttcactttcgacgttatctccggaatcttgact
gacatcagaaagatattcccattcgagttgttccacttgggaggtgacga
ggttaatactgactgttggacttccacttcccacgttaaggaatggttgt
ccactcagaacatgactgctaaggatgcttacgaatacttcgttttgaag
gctcaagagatcgctgtttctaagaactggtcccctgttaactgggaaga
gactttcaacactttcccagctaagttgcacaagaaaactgttgttcaca
actggttgggtccaggtgtttgtccaaaggttgttgctaagggtttcaga
tgtatcttctccaaccagggtgtttggtacttggaccacttggatgttcc
ttgggacgaggtttacactgctgaaccattggaaggtatcgagaagtcct
ctgagcaagagttggttatcggtggtgaagtttgtatgtggggtgagact
gctgacacttctaacgttcagcagactatctggccaagagccgcagctgc
tgctgaaagattgtggtcccaaagagactccactaacatcactgttactg
ctttgccaagattgcagaacttcagatgtttgttgaacaagagaggtgtt
gctgctgctccagttaagaactactacgctagaagagccccatccggtcc
aggttcttgttacgaacaa

SEQ ID NO: 2 may further comprise a start codon (ATG) at the 5′ end and one or more stop codons at the 3′ end.

Upon expression of the polypeptide in the host cell, the polypeptide may be further processed. For example, the polypeptide may be processed into two subunits, wherein the first subunit comprises amino acids 35 to 100 of SEQ ID NO: 16 and the second subunit comprises amino acids 110 to 553 of SEQ ID NO: 16. The start and end of the first subunit and start of second subunit may vary slightly. For example, subunits comprising amino acids 34 to 101 were also detected.

Further, the polypeptide may be hexosylated and/or glycosylated. For example, the first subunit may be hexosylated.

In one embodiment, the polypeptide having a sequence as shown in SEQ ID NO: 16 is encoded by a polynucleotide having a sequence shown in SEQ ID NO: 17. The sequence is as follows:

| | |
|---|---|
| 1 | ATGTTTCTGT GCATACCCAG ATGGTTCTCT TCACCTCTTC TCATTCTCTT TGTCATTTAC |
| 61 | TGTGCCCTCT TTGCTCCTCA AGCTGCTTCT GCCACACTCA AATCTATCAT TGAACCCACT |
| 121 | GAGTCCCTCA CATACCTTTG GCCCCTCCCC GCAGACTTCA CTTCAGGCGA TGAAACTCTT |
| 181 | TCCGTTGACC CTGCACTTAC CCTCTCTGTC GCCGGCAACG GTGGTGGCTC TTCCATTCTC |
| 241 | AGAGATGCAT TTGACCGATA CAGAGGAATC ATATTCAAGC ACAGCAGTGT TGGGTTCAGT |
| 301 | CTCATAAGAA AGTTAAGGGA AAGATTGGTG TCTGTTTCTG CCTATGACAT TGCGACATTG |
| 361 | AAGATCACTG TCCATTCAGA TAACGAGGAG CTTCAACTTG GAGTGGATGA AACCTATACC |
| 421 | TTGCTGGTTC CCAAAGCCAA GGACTCTTAT GTTGCTGGGG AAGTCACAAT TGAGGCAAAC |
| 481 | ACTGTTTATG GTGCATTGCG CGGATTAGAG ACATTCAGCC AGTTGTGTTC TTTCGATTAT |
| 541 | TCGGATAAAA CAATAAAAAT ATACAAGGCA CCTTGGTCCA TCCAAGATAA ACCTAGATTT |
| 601 | TCCTATCGTG GGCTTTTGTT GGACACATCG AGGCACTATT TACCAATTAA CGTAATTAAG |
| 661 | CAGATTATTG AATCTATGTC CTATGCTAAA CTTAATGTTC TACATTGGCA CATCATAGAC |
| 721 | GAGGAGTCAT TTCCTCTTGA GGTACCTACA TATCCAAACT TGTGGAAAGG TTCATATACA |
| 781 | AAGTGGGAAC GTTACACGGT AGAAGACGCA TATGAAATTG TCAACTTCGC CAAAATGAGA |
| 841 | GGCATAAATG TGATGGCAGA AGTGGATGTT CCTGGTCATG CAGAATCATG GGGTGCTGGA |
| 901 | TATCCCAATC TTTGGCCGTC ACCTTCCTGT AGGGAGCCAC TGGATGTTTC AAAGAATTTT |
| 961 | ACTTTTGATG TCATTTCTGG TATCCTGACA GATATAAGAA AGATTTTCCC GTTTGAGCTA |
| 1021 | TTTCACTTGG GTGGTGATGA AGTTAATACA GATTGCTGGA CCAGTACTTC TCATGTGAAG |
| 1081 | GAATGGCTTT CGACTCAAAA CATGACTGCT AAAGATGCCT ATGAATATTT TGTACTGAAG |
| 1141 | GCCCAAGAGA TAGCTGTTTC AAAAAATTGG AGTCCGGTGA ACTGGGAAGA AACCTTCAAT |
| 1201 | ACATTTCCAG CAAAGCTCCA TAAGAAAACT GTGGTGCATA ACTGGTTGGG CCCTGGGGTT |
| 1261 | TGTCCAAAGG TTGTTGCAAA AGGTTTCAGG TGCATTTTCA GTAATCAGGG TGTCTGGTAT |
| 1321 | CTTGACCATC TGGATGTACC TTGGGATGAG GTCTATACTG CTGAGCCACT AGAAGGAATA |
| 1381 | GAAAAATCTT CTGAACAAGA GCTTGTAATT GGAGGAGAAG TTTGCATGTG GGGTGAGACA |
| 1441 | GCTGATACAT CCAATGTTCA GCAAACAATA TGGCCTAGAG CTGCTGCAGC TGCAGAACGC |
| 1501 | TTATGGAGTC AGAGAGATTC TACAAATATT ACTGTAACTG CGTTGCCCCG GTTACAAAAC |
| 1561 | TTCAGATGTC TATTGAATAA ACGTGGAGTT GCAGCTGCTC CTGTGAAAAA TTATTATGCT |
| 1621 | AGAAGGGCTC CTAGTGGTCC AGGCTCATGT TATGAGCAAT AA |

In one embodiment, the polynucleotide encoding a polypeptide having β-hexosaminidase activity is codon optimized for the host cell, such as for a human cell. For example, the polynucleotide may comprise a sequence shown in SEQ ID NO: 18:

| | |
|---|---|
| 1 | GCCACACTGA AGTCCATCAT CGAGCCCACC GAGAGCCTGA CCTACCTGTG GCCTCTGCCC |
| 61 | GCCGATTTCA CCAGCGGCGA CGAGACACTG TCCGTGGATC CTGCCCTGAC ACTGAGCGTG |
| 121 | GCCGGAAATG GCGGCGGAAG CAGCATCCTG AGAGATGCCT TCGACCGGTA CAGAGGCATC |
| 181 | ATCTTCAAGC ACAGCAGCGT GGGCTTCAGC CTGATCCGGA AGCTGCGCGA GAGACTGGTG |
| 241 | TCCGTGTCCG CCTACGATAT CGCCACCCTG AAGATCACCG TGCACTCCGA CAACGAGGAA |
| 301 | CTGCAGCTGG GCGTGGACGA GACATACACC CTGCTGGTGC CCAAGGCCAA GGACAGCTAT |
| 361 | GTGGCCGGCG AAGTGACCAT CGAGGCCAAC ACAGTGTACG GCGCCCTGAG AGGCCTGGAA |
| 421 | ACCTTCAGCC AGCTGTGCAG CTTCGACTAC AGCGACAAGA CCATCAAGAT CTACAAGGCC |
| 481 | CCTTGGAGCA TCCAGGACAA GCCCCGGTTC AGCTACAGAG GCCTGCTGCT GGACACCAGC |
| 541 | AGACACTACC TGCCCATCAA CGTGATCAAG CAGATCATCG AGAGCATGAG CTACGCCAAG |
| 601 | CTGAACGTGC TGCACTGGCA CATCATCGAC GAGGAATCCT TCCCACTGGA AGTGCCCACC |
| 661 | TACCCCAACC TGTGGAAGGG CAGCTACACC AAGTGGGAGC GGTACACCGT GGAAGATGCC |
| 721 | TACGAGATCG TGAACTTCGC CAAGATGCGG GGCATCAATG TGATGGCCGA GGTGGACGTG |
| 781 | CCAGGCCACG CTGAATCTTG GGGAGCCGGC TACCCTAATC TGTGGCCCAG CCCCAGCTGT |
| 841 | CGCGAACCCC TGGACGTGTC CAAGAACTTC ACCTTCGACG TGATCAGCGG CATCCTGACC |
| 901 | GATATCAGAA AGATCTTCCC ATTCGAGCTG TTCCACCTGG GAGGCGACGA AGTGAACACC |
| 961 | GACTGCTGGA CCAGCACCAG CCACGTGAAA GAGTGGCTGA GCACCCAGAA CATGACCGCC |
| 1021 | AAGGACGCCT ACGAGTACTT CGTGCTGAAG GCCCAGGAAA TCGCCGTGTC TAAGAATTGG |
| 1081 | AGCCCCGTGA ACTGGGAGGA AACCTTTAAC ACCTTCCCTG CCAAACTGCA CAAGAAAACC |
| 1141 | GTGGTGCACA ATTGGCTGGG CCCTGGCGTG TGCCCTAAGG TGGTGGCCAA GGGCTTCCGC |
| 1201 | TGCATATTCA GCAACCAGGG CGTGTGGTAT CTGGACCACC TGGATGTGCC CTGGGACGAG |
| 1261 | GTGTACACAG CCGAGCCTCT GGAAGGCATC GAGAAGTCCT CCGAGCAGGA ACTCGTGATC |
| 1321 | GGCGGAGAAG TGTGCATGTG GGGCGAGACA GCCGACACCT CCAACGTGCA GCAGACCATC |
| 1381 | TGGCCTAGAG CCGCCGCTGC CGCTGAAAGA CTGTGGTCCC AGAGAGACAG CACCAACATC |
| 1441 | ACCGTGACCG CCCTGCCCCG GCTGCAGAAC TTTAGATGCC TGCTGAACAA GCGGGGCGTG |
| 1501 | GCCGCTGCCC CCGTGAAGAA TTACTATGCC AGAAGGGCCC CCAGCGGCCC TGGCAGCTGT |
| 1561 | TATGAACAGT GA |

The polynucleotide comprising a sequence shown in SEQ ID NO: 18 encodes a polypeptide having β-hexosaminidase activity, wherein the polypeptide has a sequence as shown in SEQ ID NO: 1.

“Percent (%) amino acid sequence identity” with respect to a reference polypeptide sequence is defined as the percentage of amino acid residues in a candidate sequence that are identical with the amino acid residues in the reference polypeptide sequence, after aligning the sequences and introducing gaps, if necessary, to achieve the maximum percent sequence identity. In some embodiments, standard parameters are applied for determining the degree of sequence identity of two sequences. For example, the degree of identity is to be determined by comparing two optimally aligned sequences over a comparison window, where the fragment of amino acid sequence in the comparison window may comprise additions or deletions (e.g., gaps or overhangs) as compared to the reference sequence (which does not comprise additions or deletions) for optimal alignment. The percentage is calculated by determining the number of positions at which the identical amino acid residue occurs in both sequences to yield the number of matched positions, dividing the number of matched positions by the total number of positions in the window of comparison and multiplying the result by 100 to yield the percentage of sequence identity. Optimal alignment of sequences for comparison may be conducted by the local homology algorithm of Smith and Waterman Add. APL. Math. 2:482 (1981), by the homology alignment algorithm of Needleman and Wunsch J. Mol. Biol. 48:443 (1970), by the search for similarity method of Pearson and Lipman Proc. Natl. Acad. Sci. (USA) 85: 2444 (1988), by computerized implementations of these algorithms (GAP, BESTFIT, BLAST, PASTA, and TFASTA in the Wisconsin Genetics Software Package, Genetics Computer Group (GCG), 575 Science Dr., Madison, WI), or by visual inspection. In some embodiments, the degree of sequence identity is determined over the entire length of the sequences. Given that two sequences have been identified for comparison, GAP and BESTFIT are preferably employed to determine their optimal alignment and, thus, the degree of identity. Preferably, the default values of 5.00 for gap weight and 0.30 for gap weight length are used. In an embodiment, the percent identity between two amino acid sequences is determined using the Needleman and Wunsch algorithm (Needleman 1970, J. Mol. Biol. (48):444-453) which has been incorporated into the needle program in the EMBOSS software package (EMBOSS: The European Molecular Biology Open Software Suite, Rice, P., Longden, I., and Bleasby, A., Trends in Genetics 16(6), 276-277, 2000), a BLOSUM62 scoring matrix, and a gap opening penalty of 10 and a gap extension penalty of 0.5. A non-limiting example of parameters to be used for aligning two amino acid sequences using the needle program are the default parameters, including the EBLOSUM62 scoring matrix, a gap opening penalty of 10 and a gap extension penalty of 0.5.

The polynucleotide as referred to herein may either essentially consist of the aforementioned nucleic acid sequences or comprise the aforementioned nucleic acid sequences. Thus, they may contain further nucleic acid sequences as well.

In some embodiments, the polynucleotide encoding the polypeptide having β-hexosaminidase activity is operably linked to a promoter, such as a heterologous promoter. Typically, a promoter comprises regulatory elements which mediate the expression of a coding sequence segment in the host cell.

In one embodiment, the promoter is a constitutive promoter. In one alternative embodiment, the promoter is an inducible promoter.

A “promoter” or “promoter sequence” is a nucleotide sequence located upstream of a gene on the same strand as the gene that enables that gene's transcription. Promoter is followed by the transcription start site of the gene. A promoter is recognized by RNA polymerase (together with any required transcription factors), which initiates transcription. A functional fragment or functional variant of a promoter is a nucleotide sequence which is recognizable by RNA polymerase, and capable of initiating transcription.

An “active promoter fragment”, “active promoter variant”, “functional promoter fragment” or “functional promoter variant” describes a fragment or variant of the nucleotide sequences of a promoter, which still has promoter activity.

A promoter can be an “inducer-dependent promoter” or an “inducer-independent promoter” comprising constitutive promoters or promoters which are under the control of other cellular regulating factors.

The person skilled in the art is capable to select suitable promoters for expressing the polypeptide of interest. For example, the polynucleotide encoding the polypeptide of interest is, typically, operably linked to an “inducer-dependent promoter” or an “inducer-independent promoter”. Further, the polynucleotide encoding the polypeptide having β-hexosaminidase activity is, typically, operably linked to an “inducer-independent promoter”, such as a constitutive promoter.

An “inducer dependent promoter” is understood herein as a promoter that is increased in its activity to enable transcription of the gene to which the promoter is operably linked upon addition of an “inducer molecule” to the fermentation medium. Thus, for an inducer-dependent promoter, the presence of the inducer molecule triggers via signal transduction an increase in expression of the gene operably linked to the promoter.

In an embodiment, the promoter is a CMV promoter. For example, a CMV may be used when expressing the polypeptide having β-hexosaminidase activity in a mammalian host cell, such as a HEK-293 host cell.

In another embodiment, the promoter is a Tac promoter. For example, the Tac promoter may be used when expressing the polypeptide having β-hexosaminidase activity in a yeast host cell, such as a yeast cell disclosed herein below. The Tac-Promoter (abbreviated as Ptac), is a synthetically produced DNA promoter, produced from the combination of promoters from the trp and lac operons. It is commonly used for protein production.

In an embodiment, the promoter is the promoter of a polynucleotide encoding for an alcohol oxidase, such as a promoter from a yeast AOX1 (Alcohol Oxidase 1).

The term “operably linked”, typically refers to a functional linkage between the promoter sequence and the gene of interest (i.e. the polynucleotide encoding a polypeptide having 8-hexosaminidase activity), such that the promoter sequence is able to initiate transcription of the gene of interest.

Further, the polynucleotide as referred to herein may be operably linked to a terminator. The term “terminator” typically encompasses a control sequence which is a DNA sequence at the end of a transcriptional unit which signals 3′ processing and polyadenylation of a primary transcript and termination of transcription.

The polynucleotide as referred to herein may be further operably linked to a polynucleotide which encodes for a secretion leader, i.e. a sequence which allows for secretion of the 8-hexosaminidase of the invention into the cultivation medium.

The host cell provided in step a) of the method of the present invention may be any host cell deemed appropriate. For example, the host selected from the group consisting of bacterial cells, such as an E. coli cell, a yeast cell, an algal cell, or a plant cell. The term “host cell” further includes animal cells, such as non-human animal cells.

In some embodiments, the host cell is a eukaryotic host cell.

In some embodiments, the host cell is a yeast cell.

In some embodiments, the yeast cell belongs to the family of Saccharomycetaceae which is a family of yeasts in the order Saccharomycetales that reproduce by budding. In some embodiments, the family of Saccharomycetaceae includes the following genera: Candida, Kluyveromyces, Komagataella, Kuraishia, Lachancea, Nakaseomyces, Pichia, Saccharomyces, Spathaspora, Tetrapisispora, Zygosaccharomyces and Zygotorulaspora.

In some embodiments, the yeast cell belongs to the genus of Kluyveromyces. For example, the yeast cell may be a Klyveromyces lactis cell.

In some embodiments, the yeast cell belongs to the genus of Pichia. For example, the yeast cell may be a Pichia pastoris cell.

In some embodiments, the yeast cell belongs to the genus of Komagataella. For example, the yeast cell may be a Komagataella phaffii cell, such as a cell of the Komagataella phaffii strain ATCC 76273. More information on this strain can be found in the UniProt database (see Taxon Identifier 981350).

In some embodiments, the host cell is not a Canavalia ensiformis cell.

In some embodiments, the host cell is a mammalian host cell. Suitable mammalian cells include, but are not limited to, for example, CHO (Chinese Hamster Ovary) cells, BHK cells, HeLa cells, COS cells, HEK-293 and the like. In one embodiment, HEK-293 cells are used. In another embodiment, CHO cells are used.

Step b) of the method of the present invention comprises cultivating the host cell under conditions which allow for the production, i.e. the production of the polypeptide having β-hexosaminidase activity. Such conditions are well-known in the art and, e.g., described in the Examples section.

The method of the present invention may further comprise step c) of obtaining the polypeptide produced in step b). Said polypeptide shall be obtained from the cultivation medium by methods known in the art.

The present invention further relates to a polynucleotide as defined herein above in connection with the method of the present invention, i.e. a polynucleotide encoding a polypeptide having β-hexosaminidase activity and having an amino acid sequence being at least 85% identical to the amino acid sequence shown in SEQ ID NO: 1.

Further encompassed by the present invention is an isolated polypeptide encoded by the polynucleotide of the present invention. The polypeptide has been defined above. The isolated polypeptide may be hexosylated and/or glycosylated.

The present invention further pertains to a host cell comprising the polynucleotide of the present invention, the polypeptide of the present invention and/or the vector of the present invention.

Moreover, the present invention is directed to a vector comprising the polynucleotide of the present invention. In some embodiments, the vector is an expression vector.

The term “vector”, typically, encompasses phage, plasmid, viral or retroviral vectors as well as artificial chromosomes, such as bacterial or yeast artificial chromosomes. Moreover, the term also relates to targeting constructs which allow for random or site-directed integration of the targeting construct into genomic DNA. Such target constructs, preferably, comprise DNA of sufficient length for either homologous or heterologous recombination as described in detail below. The vector encompassing the polynucleotides of the present invention, preferably, further comprises selectable markers for propagation and/or selection in a host. The vector may be incorporated into a host cell by various techniques well known in the art. If introduced into a host cell, the vector may reside in the cytoplasm or may be incorporated into the genome. In the latter case, the vector may further comprise nucleic acid sequences which allow for homologous recombination or heterologous insertion. Vectors can be introduced into prokaryotic or eukaryotic cells via conventional transformation or transfection techniques. The terms “transformation” and “transfection”, conjugation and transduction, as used in the present context, are intended to comprise a multiplicity of prior-art processes for introducing foreign nucleic acid (for example DNA) into a host cell, including calcium phosphate, rubidium chloride or calcium chloride co-precipitation, DEAE-dextran-mediated transfection, lipofection, natural competence, carbon-based clusters, chemically mediated transfer, electroporation or particle bombardment (e.g., “gene-gun”). Suitable methods for the transformation or transfection of host cells, including yeast cells, can be found in Sambrook et al. (Molecular Cloning: A Laboratory Manual, 2^{nd} ed., Cold Spring Harbor Laboratory, Cold Spring Harbor Laboratory Press, Cold Spring Harbor, N Y, 1989) and other laboratory manuals, such as Methods in Molecular Biology, 1995, Vol. 44, Agrobacterium protocols, Ed.: Gartland and Davey, Humana Press, Totowa, New Jersey. Alternatively, a plasmid vector may be introduced by heat shock or electroporation techniques.

In some embodiments, the vector referred to herein is suitable as a cloning vector, i.e. replicable in microbial systems, such as in E. coli or in yeast cells.

Further, it is envisaged that the vector of the present invention is an expression vector. In such an expression vector, the polynucleotide comprises an expression cassette as specified above allowing for expression in a host cell. An expression vector may, in addition to the polynucleotide of the invention, also comprise further regulatory elements such as a promoter (e.g. a promoter as described elsewhere herein). Preferably, the expression vector is also a gene transfer or targeting vector.

### LIST OF EMBODIMENTS

1. A method of producing a polypeptide having β-hexosaminidase activity, comprising the steps of
   a) providing a yeast cell comprising a polynucleotide encoding a polypeptide having β-hexosaminidase activity and having an amino acid sequence being at least 95% identical to the amino acid sequence shown in SEQ ID NO: 1 or 16,
   b) cultivating said yeast cell under conditions which allow for the production of the polypeptide, and
   c) obtaining the polypeptide produced in step b).
2. The method of embodiment 1, wherein the polypeptide having β-hexosaminidase activity has an amino acid sequence which is at least 98% identical to the amino acid sequence shown in SEQ ID NO: 1.
3. The method of embodiments 1 and 2, wherein the polypeptide having 8-hexosaminidase activity comprises an amino acid sequence as shown in SEQ ID NO: 1.
4. The method of embodiments 1 to 3, wherein the yeast cell belongs to the family of Saccharomycetaceae.
5. The method of embodiment 4, wherein the yeast cell is a Komagataella cell, such as a Komagataella phaffii cell, such as a cell of the Komagataella phaffii strain ATCC
6. The method of any one of embodiments 1 to 5, wherein the polynucleotide encoding a polypeptide having β-hexosaminidase activity is operably linked to a heterologous promoter.
7. The method of any one of embodiments 1 to 6, wherein the polynucleotide encoding a polypeptide having β-hexosaminidase activity is codon optimized for the yeast cell.
8. The method of any one of embodiments 1 to 7, wherein the polynucleotide comprises a nucleic acid sequence as shown in SEQ ID NO: 2 or 17.
9. A polynucleotide encoding a polypeptide having β-hexosaminidase activity and having an amino acid sequence being at least 95% identical to the amino acid sequence shown in SEQ ID NO: 1.
10. The polynucleotide of embodiment 9, wherein the polypeptide having β-hexosaminidase activity comprises an amino acid sequence as shown in SEQ ID NO: 1.
11. The polynucleotide of embodiment 9, wherein the polynucleotide is operably linked to a heterologous promoter.
12. A vector, such as an expression vector, comprising the polynucleotide of any one of embodiments 9 to 11.
13. A yeast cell comprising the polynucleotide of any one of embodiments 9 to 11, or the vector of embodiment 12.
14. The yeast cell of embodiment 13, wherein the yeast cell belongs to the family of Saccharomycetaceae.
15. The yeast cell of embodiment 14, wherein the yeast cell is Komagataella phaffii.
16. An isolated polypeptide encoded by the polynucleotide of any one of embodiments 9 to 11.

The following examples merely illustrate the invention. They should, whatsoever, not be construed as limiting the scope of protection.

### EXAMPLES

### Example 1: Introduction

In the studies underlying the present invention, the mRNA sequence and the protein sequence of β-hexosaminidase from Canavalia ensiformis were determined. First, the sequence of portions of a β-hexosaminidase protein that has been extracted from Canavalia ensiformis plants were determined by preparative digest, MS/MS and N-terminal sequencing. Subsequently, the cDNA sequence was determined by 3′ and 5′ RACE (Rapid amplification of cDNA ends).

A database research in NCBI and KEGG resulted in the mRNA derived sequences of 4 β-hexosaminidases from Glycine max (soybean), the next sequenced relative of C. ensiformis (Jack Bean) with the following NCBI IDs:
Chromosome 2 (1668 nt); cDNA XM_003518614.2; protein XP_003518662.1
Chromosome 10 (1632 nt); cDNA XM_003535730.2; protein XP_003535778.1
Chromosome 18 (1698 nt); cDNA XM_003552624.2; protein XP_003552672.1
Chromosome 20 (1641 nt); cDNA XM_003555573.2; protein XP_003555621.1

These sequences served as a basis for primer design and comparison of elucidated sequences.

### Example 2: Determination of Portions of the Protein Sequence of a β-Hexosaminidase from Canavalia ensiformis

β-hexosaminidase purified from Canavalia ensiformis (and having an apparent molecular weight of ˜55 kDa) was digested with Lys-C and the resulting peptides were separated via HPLC. After this, Edman degradation was performed with the fractions.

100 μl β-hexosaminidase (˜77 μg) were vortexted with 29 mg guanidinium hydrochloride (solid) to reach a final concentration of ˜3 M GuaHCl. 7 μl 1.5 M Tris/HCl pH 8.8 were added and shortly vortexed again. 3 μl were taken out for testing the pH on strips (˜pH 8.5). The remaining solution was denatured for 20 min at −80° C. and shock cooled in an ice bath.

One vial Lys-C(5 μg, Roche Cat. 11047825001) was reconstituted with 50 μl of water. 5 μl (0.5 μg Lys-C) of this solution were added to the shock cooled solution, vortexed again and incubated at 32° C. for 3 h. 95 μl were directly injected on an Agilent 1200 HPLC equipped with fraction collector using a Waters column (X-Select CSH C18 2.5 μm 2.1×150 mm, Cat. 186006727). Chromatographic separation resulted in sharp peaks with fraction volumes of 100 to 150 μl containing ˜25% ACN solvent. 36 fractions were collected (not shown). These were directly used for MALDI-MS to determine the peptide mass (e.g. to estimate the number of Edman cycles).

For some of the obtained fractions the amino acid sequence could be determined by N-terminal Edman sequencing using either an Applied Biosystems Procise HT or Shimadzu PPSQ-33A sequencer under standard conditions. The number of cycles (=amino acid) for each fraction was estimated by a MALDI-MS measurement.

Edman degradation of the fractions resulted in a multitude of sequences that were aligned using ClustalW. In total 208 out of 553 amino acids were identified. The de novo sequenced peptides were superposed to the translated cDNA sequence that was identified as described in Example 3 below (not shown). The results show that the right cDNA sequence was identified in C. ensiformis.

### Example 3: Determination of the cDNA Sequence of a β-Hexosaminidase from Canavalia Ensiformis

Jack Bean (C. ensiformis) seeds were put in between wet absorbent tissue into a plastic tray and stored for about 48 hours at a dark place at room temperature (for germination). Afterwards, the germinated seeds were grown for another 5-6 days at light at room temperature. Then the small plants were put into Vermiculite, 3-6 mm as substrate (in 2-3 cm depth) at a sunny window at room temperature and watered if dry.

Germinated material from C. ensiformis was cut with a scalpel into parts which can be used for RNA extraction (about 200 mg plant material), put into 50 ml plastic tubes and snap frozen in liquid nitrogen. This was performed with sprout, cotyledon, embryo and leaf tissue. Isolation of RNA from the aforementioned tissues was done according to manufacturer's instructions (RNeasy Plant Mini Kit (Qiagen Cat #74903))

cDNA was synthesized with two reverse transcriptases for sprout, cotyledon, embryo and leaf respectively. Then, cDNAs of both reverse transcriptase reactions for sprout, cotyledon, embryo and leaf respectively were pooled.

Subsequently, an internal fragment of each cDNA was amplified by PCR using Phusion Hot Start II DNA Polymerase (Thermo Scientific, Cat #F-549L) and the following primers:

JB-01
(SEQ ID NO: 3)
CTCACCTACCTCTGGCCCCTTCCCGC
JB-07
(SEQ ID NO: 4)
TTATTGGTCATAACATGACCCTGGACCAACAGG

Afterwards, the amplified fragments were subjected to DNA sequence analysis using the Big Dye® Cycle Sequencing Terminator Kit (Applied Biosystems, USA) and the following primers:

JB-01
(SEQ ID NO: 3)
CTCACCTACCTCTGGCCCCTTCCCGC
JB-02
(SEQ ID NO: 5)
GAGGAGCTTCAATTTGGAGTGGATG
JB-06
(SEQ ID NO: 6)
ATCAGCTGTCTCACCCCACATGCAAACTTCTC
JB-07
(SEQ ID NO: 4)
TTATTGGTCATAACATGACCCTGGACCAACAGG,

About 100 ng PCR fragment (or 300 ng plasmid DNA) and 10 pmol primer were amplified with the Big Dye® Cycle Sequencing Terminator Kit, with DyeEX 2.0 Spin Kit purified and sequenced. The Kits and equipment were used according to manufacturer's instructions.

Afterwards, a 3′RACE and 5′RACE were carried out with cDNA obtained from cotyledon tissue.

The following primers were used

For the 3′RACE:

JB-08
(SEQ ID NO: 7)
AAGTTTGCATGTGGGGTGAGAC
JB-09
(SEQ ID NO: 8)
GCAAACAATATGGCCTAGAGCTG
CDSIII-short
(SEQ ID NO: 9)
ATTCTAGAGGCCGAGGCGGCCGACATGT

Two PCRs, one with JB-08+CDSIII-short and one with JB-09+CDSIII-short were performed. PCR fragments were sequenced using the JB-09 primer.

For the 5′RACE:

JB-10
(SEQ ID NO: 10)
AAGAGTCCTTGGCTTTGGGAAC
Okib57-Adapter
(SEQ ID NO: 11)
5′-pGTAGGAATTCGGGTTGTAGGGAGGTCGACATTGCC-3′
JB-01
(SEQ ID NO: 3)
CTCACCTACCTCTGGCCCCTTCCCGC
JB-11
(SEQ ID NO: 12)
TCAATGTCGCAATGTCATAGGC
JB-12
(SEQ ID NO: 13)
ATGAGACTGAACCCAACACTGC
Okib58
(SEQ ID NO: 14)
5′-GGCAATGTCGACCTCCCTACAAC-3′
Okib59
(SEQ ID NO: 15)
5′-CTCCCTACAACCCGAATTCCTAC-3′

cDNA was synthesized with both transcriptases for cotyledon with the specific primer JB-10. Then both cDNAs were pooled. Okib57-Adapter was ligated to the freshly synthesized cDNA. One PCR was performed with primers JB-11 and Okib58 and one PCR was performed with primers JB-12 and Okib59. The resulting fragment was subcloned in PCR-Blunt-II-TOPO and sequenced as described above.

In summary, the mRNA sequence of β-hexosaminidase from Canavalia ensiformis was successfully obtained. mRNA could be isolated from different freshly germinated plant material. The corresponding cDNA was sequenced and the found sequence was confirmed by the partial elucidation of the protein sequence of β-hexosaminidase (purified β-hexosaminidase).

### Example 4: Recombinant Expression of the Identified Polypeptide

The beta-Hexosaminidase from Canavalia ensiformis was recombinantly expressed under control of an AOX1 promoter in Komagataella phaffii strain ATCC 76273 (also referred to as CBS 7435). For recombinant expression of beta-Hexosaminidase in 96-deep well plates single colonies were picked from transformation plates into single wells of 96-deep well plates filled with optimized cultivation media. After an initial growth phase to generate biomass, expression from the AOX1 promoter was induced by addition of an optimized liquid mixture allowing for derepressive expression. After a total of 108 hours from the initial inoculation, all deep well plates were centrifuged and supernatants of all wells were harvested into stock microtiter plates for subsequent analysis.

For recombinant expression of beta-Hexosaminidase in fermentation scale, 50 mL of Yeast/Peptone/Glycerol medium in 300 mL shake flask were inoculated with the production strain and shaken at 110 rpm at 28° C. over night (pre-culture 1). Pre-culture 2 (200 mL Yeast/Peptone/Glycerol medium in a 2 L shake flask) was inoculated from pre-culture 1 in that way that the OD600 nm reached approximately 20. Pre-culture 2 was shaken at 220 rpm at 28° C. for about 8 h. 2 L fermenters filled with 400 mL defined medium containing glycerol as carbon source (pH=5.5) were inoculated from pre-culture 2 to an OD600 nm of 2.0, during initial batch phase cultivation temperature was 28° C. One hour prior initiating the production phase temperature was decreased to 24° C. and kept at this level throughout the remaining process, while the pH dropped to 5.0 and was kept at this level. Oxygen saturation was set to 30% throughout the whole process (cascade control: stirrer, flow, oxygen supplementation). Stirring was applied between 700 and 1200 rpm and a flow range (air) of 1.0-2.0 L min-1 was chosen. Glycerol fed-batch was performed by supplying a 60% glycerol solution at 6 g/L h throughout the whole cultivation.

1. A method of producing a polypeptide having β-hexosaminidase activity, comprising the steps of
a) providing a yeast cell comprising a polynucleotide encoding a polypeptide having β-hexosaminidase activity and having an amino acid sequence being at least 95% identical to the amino acid sequence shown in SEQ ID NO: 1 or 16,
b) cultivating said yeast cell under conditions which allow for the production of the polypeptide, and
c) obtaining the polypeptide produced in step b).

2. The method of claim 1, wherein the polypeptide having β-hexosaminidase activity has an amino acid sequence which is at least 98% identical to the amino acid sequence shown in SEQ ID NO: 1.

3. The method of claim 1, wherein the polypeptide having β-hexosaminidase activity comprises an amino acid sequence as shown in SEQ ID NO: 1.

4. The method of claim 1, wherein the yeast cell belongs to the family of Saccharomycetaceae.

5. The method of claim 1, wherein the yeast cell is a Komagataella cell.

6. The method of claim 1, wherein the polynucleotide encoding a polypeptide having β-hexosaminidase activity is operably linked to a heterologous promoter.

7. The method of claim 1, wherein the polynucleotide encoding a polypeptide having β-hexosaminidase activity is codon optimized for the yeast cell.

8. The method of claim 1, wherein the polynucleotide comprises a nucleic acid sequence as shown in SEQ ID NO: 2 or 17.

9. A polynucleotide encoding a polypeptide having β-hexosaminidase activity and having an amino acid sequence being at least 95% identical to the amino acid sequence shown in SEQ ID NO: 1.

10. The polynucleotide of claim 9, wherein the polypeptide having β-hexosaminidase activity comprises an amino acid sequence as shown in SEQ ID NO: 1.

11. The polynucleotide of claim 9, wherein the polynucleotide is operably linked to a heterologous promoter.

12. A vector, such as an expression vector, comprising the polynucleotide of claim 9.

13. A yeast cell comprising the polynucleotide of claim 9.

14. The yeast cell of claim 13, wherein the yeast cell belongs to the family of Saccharomycetaceae.

15. An isolated polypeptide encoded by the polynucleotide of claim 9.

16. A yeast cell comprising the vector of claim 12.

17. The method of claim 3, wherein the yeast cell belongs to the family of Saccharomycetaceae.

18. The method of claim 3, wherein the yeast cell is a Komagataella cell.

19. The method of claim 18, wherein the Komagataella cell is a Komagataella phaffii cell.

20. The method of claim 5, wherein the Komagataella cell is a Komagataella phaffii cell.
